(19) United States
(12) Patent Application Publication
(10) Pub. No.: US 2016/0199636 A1
(43) Pub. Date: Jul. 14, 2016

(54) SILICONE CONCENTRIC ELECTRODES AND ELECTRICAL STIMULATION SYSTEM

(71) Applicant: NATIONAL YANG-MING UNIVERSITY, Taipei (TW)

(72) Inventors: Ming-Chang Chiang, Taipei (TW); Ssu-Min Chang, Taipei (TW); Shung-Fu Wang, Taipei (TW)

(21) Appl. No.: 14/687,605

(22) Filed: Apr. 15, 2015

(30) Foreign Application Priority Data
Jan. 8, 2015 (TW) 104100507

(51) Int. Cl. A61N 1/04

(52) U.S. Cl. CPC A61N 1/0472

(57) ABSTRACT

The present disclosure illustrates a silicone concentric electrode and applies to electrical stimulation. The electrode includes at least a silicone electrode sheet and two wires. The silicone electrode sheet has a non-conductive rubber that separates the positive electrode and the negative electrode, and the negative electrode and the positive electrode will be connected to each wire respectively.

### BACKGROUND OF THE INVENTION

1. Field of the Invention

The present disclosure generally relates to a silicone concentric electrode and an electrical stimulation system thereof which can perform electrical stimulation during magnetic resonance image scanning without interfering the imaging process of the magnetic resonance image scanning, such that the functional magnetic resonance image scanning can be performed to observe changes in each of the brain areas.

2. Description of the Related Art

Clinically, in order to detect small-nerve-fiber disease such as diabetes or AIDS, the electrical stimulation is often executed on patients to observe patient's somatosensory evoked potential (SSEP) and nerve conduction velocity (NCV), and the results of these test items are used to determine whether the small nerve fibers are normal. A small nerve fiber indicates an Aδ or C fiber, while a large nerve fiber indicates an Aβ fiber. The stimulation executed by using the traditional electrode sheet usually causes the small and large nerve fibers to be activated at the same time, but hardly just activates the small nerve fiber exclusively, such that physiological signal will be subjected to interferences. In such a situation, it will cause false results of clinical diagnosis or confusion of experiment results.

In addition, in various studies exploring pain sense, the electrical stimulation is used to give stabbing pain to testees. The Aβ fiber belonging to the large nerve fiber is responsible for proprioception or tactile sensation. If the small and large nerve fibers of the testee are activated at the same time by using the traditional electrode, the testee can feel stabbing pain, heat pain, tactile sensation and other senses at the same time, but the testee cannot precisely feel the stabbing pain caused by activation of the small nerve fiber, and in such situation the experiment usually results an error.

In various electrical stimulation experiments and clinical conditions, the electrical stimulator is used for applying electrical stimulation to human body, whereas the medium conducting current between the electrical stimulator and human body is an electrode. The electrode paster is often used as the electrode during the electrical stimulation process. At present, common electrode paster is a button-type electrode or a metal electrode, and these two electrodes both have a great number of ferromagnetic metals. The ferromagnetic metal can result in an uneven distribution of the magnetic field in magnetic resonance imaging (MRI), such that the imaging process of magnetic resonance imaging may be interfered. Thus, the magnetic resonance imaging scanning and the electrical stimulation cannot be carried out at the same time, so the functional magnetic resonance imaging (fMRI) scanning cannot be performed to observe changes in each encephalic region of the brain for further exploring the association of diseases and brain regions. In addition, the ferromagnetic metal has excellent thermal conductivity and hence human skin may be burned during the electrical stimulation process.

### SUMMARY OF THE INVENTION

An objective of the present disclosure is to improve the material and structure of electrode, such that the electrode is able to perform magnetic resonance imaging scanning and electrical stimulation at the same time without the problems of non-uniform magnetic field distribution, the imaging process being interfered, and human skin being burned, etc.

According to the objective, the present disclosure provides a silicone concentric electrode applied to electrical stimulation. The silicone concentric electrode includes a silicone electrode sheet and two wires. The silicone electrode sheet has a positive electrode and a negative electrode which are connected to different wires respectively.

In the embodiment, the silicone electrode sheet has a non-conductive rubber thereon to separate the positive electrode and the negative electrode.

In the embodiment, the two wires are connected to the positive electrode and the negative electrode by being adhered with the conductive silver adhesive.

According to the objective, the present disclosure provides a silicone concentric electrode applied to electrical stimulation. The silicone concentric electrode includes a circular silicone electrode sheet, a ring-like silicone electrode sheet and two wires. The circular silicone electrode sheet and ring-like silicone electrode sheet have a positive electrode and a negative electrode corresponding to each other. The positive electrode and the negative electrode are connected to different wires respectively.

In the embodiment, a non-conductive rubber is arranged between the circular silicone electrode sheet and the ring-like silicone electrode sheet to separate the positive electrode and the negative electrode.

In the embodiment, the two wires can be connected to the circular silicone electrode sheet and ring-like silicone electrode by being adhered with the conductive silver adhesive.

According to the objective, the present disclosure provides an electrical stimulation system including a silicone electrode sheet, two wires and an electrical stimulation. The silicone electrode sheet has the positive electrode and the negative electrode which are connected to different wires respectively. The electrical stimulation is electrically connected to two wires and supplied necessary frequency current from the wires to the silicone electrode sheet.

In the embodiment, the silicone electrode sheet is provided with a non-conductive rubber thereon to separate the positive electrode and the negative electrode.

In the embodiment, the two wires are connected to the positive electrode and the negative electrode by being adhered with the conductive silver adhesive.

### BRIEF DESCRIPTION OF THE DRAWINGS

The detailed structure, operating principle and effects of the present disclosure will now be described in more details hereinafter with reference to the accompanying drawings that show various embodiments of the present disclosure as follows.

FIG. 1 is an overall structural schematic view of the electrode according to the present disclosure.

FIG. 2 is a front schematic structural view of the electrode according to the present disclosure.

FIG. 3 is a front schematic structural view of the radius of the electrode according to the present disclosure.

FIG. 4 is a schematic view of the electrical stimulation system.

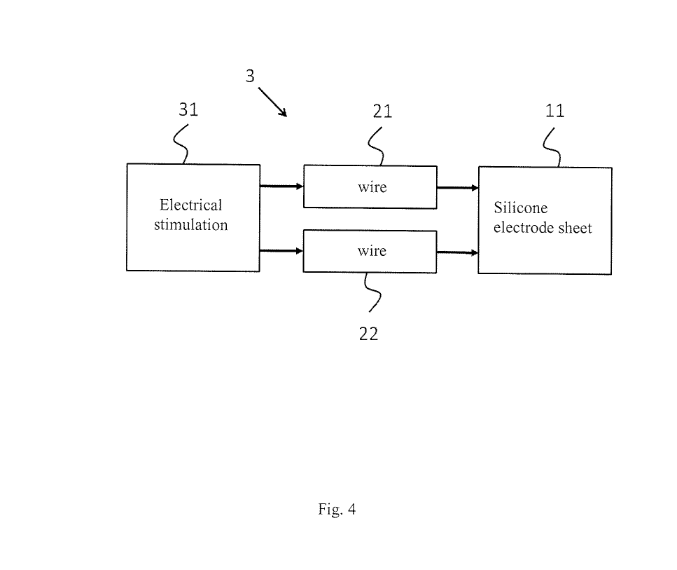

### DETAILED DESCRIPTION OF THE PREFERRED EMBODIMENTS

Reference will now be made in detail to the exemplary embodiments of the present disclosure, examples of which are illustrated in the accompanying drawings. Therefore, it is to be understood that the foregoing is illustrative of exemplary embodiments and is not to be construed as limited to the specific embodiments disclosed, and that modifications to the disclosed exemplary embodiments, as well as other exemplary embodiments, are intended to be included within the scope of the appended claims. These embodiments are provided so that this disclosure will be thorough and complete, and will fully convey the inventive concept to those skilled in the art. The relative proportions and ratios of elements in the drawings may be exaggerated or diminished in size for the sake of clarity and convenience in the drawings, and such arbitrary proportions are only illustrative and not limiting in any way. The same reference numbers are used in the drawings and the description to refer to the same or like parts.

It will be understood that, although the terms ‘first’, ‘second’, ‘third’, etc., may be used herein to describe various elements, these elements should not be limited by these terms. The terms are used only for the purpose of distinguishing one component from another component. Thus, a first element discussed below could be termed a second element without departing from the teachings of embodiments. As used herein, the term “or” includes any and all combinations of one or more of the associated listed items.

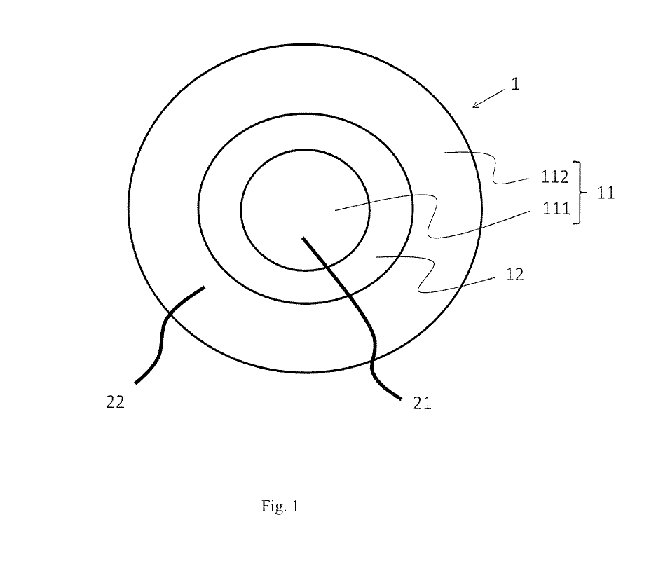

In the embodiment, a silicone concentric electrode 1 applied to electrical stimulation, of which structure includes a silicone electrode sheet 11 and two wires 21 and 22. The silicone electrode sheet 11 has a non-conductive rubber 12 that separates a negative electrode and a positive electrode. The negative and positive electrodes are connected to different wires 21 and 22 respectively, as shown in FIG. 1.

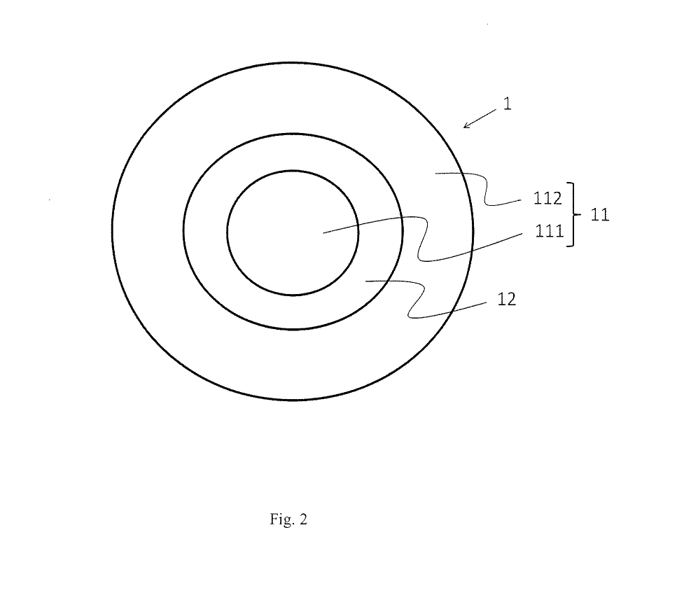

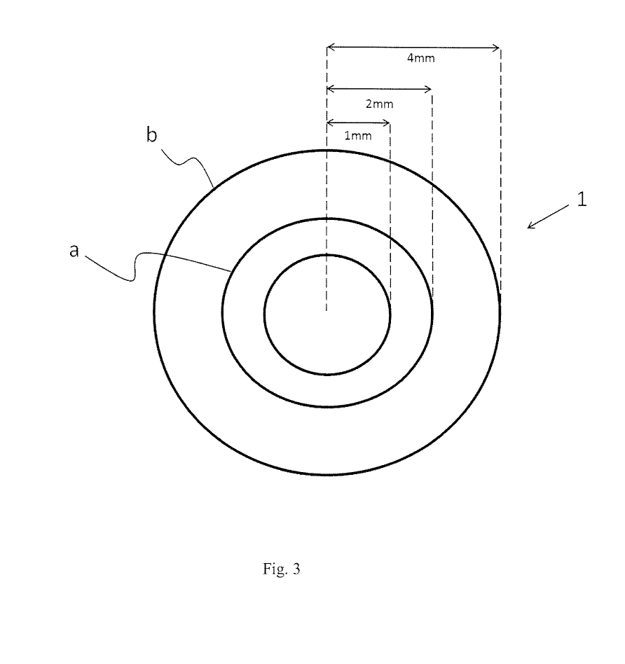

In the embodiment, a front surface of the silicone concentric electrode is configured to contact with the human skin, and the front surface is defined as a surface of silicone electrode sheet 11 which is not connected to the two wires 21 and 22, as shown in FIG. 2. The negative and positive electrodes of the silicone electrode sheet 11 are arranged in concentric circles. The negative electrode is a circular conductive silicone sheet 111 having a radius of at least one millimeter. The positive electrode is a ring-like conductive silicone sheet 112 having concentric circles of inner radius of at least two millimeters and of outer radius b of at least four millimeters. The positive and negative electrodes are separated by the non-conductive rubber 12 such that the distance between the positive electrode and the negative electrode is one millimeter. Thus, the current can just be flowed through epidermis of skin to optionally stimulate A-δ nerve fiber inside epidermis without stimulating a deep nerve fiber associated with non-pain, as shown in FIG. 3.

In the embodiment, the back surface of the silicone concentric electrode 1 is defined as a surface of the silicone electrode sheet 11 connected to two wires 21 and 22, as shown in FIG. 1. The wire 21 is connected to the circular conductive silicone sheet 111 and another wire 22 is connected to the ring-like conductive silicone sheet 112.

In the embodiment, the two wires 21 and 22 are connected on the surfaces of silicone electrode sheet 11, or buried in the silicone electrode sheet 11, or passed through silicone electrode sheet 11. The foregoing definition for the connection mode is not limited to the two wires 21 and 22 both being connected by the same way. The silicone electrode sheet 11 can be connected to one of the wires 21 and 22 by using the one of foregoing connection modes. Similarly, the silicone electrode sheet 11 is also connected to the other of the wires 21 and 22 by using the one of foregoing connection modes. For example, the wire 21 is connected on the surfaces of the silicone electrode sheet 11 and another wire 22 is buried in the silicone electrode sheet 11.

In the embodiment, the two wires 21 and 22 can be a coaxial cable resisting electromagnetic interference.

In the embodiment, the electrical stimulation system 3 includes the foregoing silicone electrode sheet 11 and two wires 21 and 22. Also, the electrical stimulation 3 further includes an electrical stimulator 31.

The electrical stimulator 31 is electrically connected to the two wires 21 and 22 to supply necessary frequency current to the silicone electrode sheet from the wires 21 and 22 to the silicone electrode sheet 11, so as to stimulate the small nerve fibers to generate a sensory signal, such as stabbing pain, etc.

In the embodiment, the electrical stimulator 31 can be a Stimulus Isolator and the other electrical stimulation products developed by AD INSTRUMENTS Company to perform the electrical stimulation. The electrical stimulator is a mature product and developed in various types, so the detailed description is omitted in the present disclosure.

In summary, the silicone concentric electrode 1 of the present disclosure does not influence distribution of magnetic field in functional magnetic resonance imaging. In addition, the thermal conductivity of the silicone electrode sheet 11 is lower than that of the metal, so the problem of burning the human skin will not be occurred.

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

What is claimed is:

1. A silicone concentric electrode, applied to, the electrode comprising:
a silicone electrode sheet, having a positive electrode and a negative electrode;
two wires, connected to the positive electrode and the negative electrode respectively.

2. The silicone concentric electrode of claim 1, wherein the silicone electrode sheet has a non-conductive rubber which separates the positive electrode and the negative electrode.

3. The silicone concentric electrode of claim 1, wherein the two wires are connected on the surfaces of the silicone electrode sheet, or buried in the silicone electrode sheet, or passed through the silicone electrode sheet.

4. The silicone concentric electrode of claim 3, wherein the two wires can be connected to the positive electrode and the negative electrode by being adhered with conductive silver adhesive.

5. A silicone concentric electrode, applied to electrical stimulation, the electrode comprising:
a circular silicone electrode sheet, having a positive electrode or a negative electrode thereon;
a ring-like silicone electrode sheet, having a negative electrode or a positive electrode thereon corresponding to the circular silicone electrode sheet; and
two wires, connected to the circular silicone electrode sheet and the ring-like silicone electrode sheet respectively.

6. The silicone concentric electrode of claim 5, wherein the positive electrode and the negative electrode of the circular silicone electrode sheet and the ring-like silicone electrode sheet are separated by a non-conductive rubber.

7. The silicone concentric electrode of claim 5, wherein the two wires are connected on the surfaces of the circular silicone electrode sheet and the ring-like silicone electrode sheet, or buried in the circular silicone electrode sheet and the ring-like silicone electrode sheet, or passed through the circular silicone electrode sheet and the ring-like silicone electrode sheet.

8. The silicone concentric electrode of claim 7, wherein the two wires can be connected to the circular silicone electrode sheet and ring-like silicone electrode by being adhered with the conductive silver adhesive.

9. An electrical stimulation system, comprising:
a silicone electrode sheet, having a positive electrode and a negative electrode;
two wires, connected to the positive electrode and the negative electrode of the silicone electrode sheet respectively; and
an electrical stimulator, electrically connected to the two wires, and supplied necessary frequency current to the silicone electrode sheet.

10. The electrical stimulation system of claim 9, wherein the positive electrode and the negative electrode of the silicone electrode sheet are separated by a non-conductive rubber.

11. The silicone concentric electrode of claim 9, wherein the two wires are connected on the surfaces of the silicone electrode sheet, or buried in the silicone electrode sheet, or passed through the silicone electrode sheet.

12. The silicone concentric electrode of claim 11, wherein the two wires can be connected to the positive electrode and the negative electrode by being adhered with the conductive silver adhesive.
